SEQUENCE LISTING (11 sequences)

SEQ ID NO: 1 - protein, 18 aa, Homo sapiens
  MISC_FEATURE (2)..(7): X can be any amino acid
WXXXXXXWGQGTLVTVSS

SEQ ID NO: 2 - protein, 17 aa, Homo sapiens
  MISC_FEATURE (2)..(8): X can be any amino acid
WXXXXXXXWGQGTTVTV

SEQ ID NO: 3 - protein, 16 aa, Homo sapiens
  MISC_FEATURE (2)..(12): X can be any amino acid
WXXXXXXXXXXXWGQG

SEQ ID NO: 4 - protein, 19 aa, Homo sapiens
  MISC_FEATURE (2)..(6): X can be any amino acid
WXXXXXWGQGTLVTVSSAS

SEQ ID NO: 5 - protein, 17 aa, Homo sapiens
  MISC_FEATURE (2)..(9): X can be any amino acid
WXXXXXXXXWGQGILVT

SEQ ID NO: 6 - protein, 18 aa, Homo sapiens
  MISC_FEATURE (2)..(8): X can be any amino acid
WXXXXXXXWGQGTLVTVS

SEQ ID NO: 7 - protein, 30 aa, Homo sapiens
TAVYYCSRWGGDGFYAMDYWGDGTLVTVSS

SEQ ID NO: 8 - protein, 28 aa, Homo sapiens
  MISC_FEATURE (2)..(15): X can be any amino acid
MXXXXXXXXXXXXXXWGQGTLVTVSSAS

SEQ ID NO: 9 - protein, 22 aa, Homo sapiens
  MISC_FEATURE (2)..(9): X can be any amino acid
MXXXXXXXXWGQGTTLTVSSAS

SEQ ID NO: 10 - protein, 15 aa, Homo sapiens
  MISC_FEATURE (2)..(6): X can be any amino acid
MXWXXXWGQGTLVTV

SEQ ID NO: 11 - protein, 22 aa, Homo sapiens
  MISC_FEATURE (2)..(10): X can be any amino acid
WXXXXXXXXXWGQGTMVTVSSA

(19) United States
(12) Patent Application Publication
(10) Pub. No.: US 2010/0173418 A1
(43) Pub. Date: Jul. 8, 2010

(54) Polypeptides with Reduced Susceptibility to Oxidation and Methods of Making

(75) Inventor: Joe Zhou, Westlake Village, CA (US)

Correspondence Address: AMGEN INC., MAIL STOP 28-2-C, ONE AMGEN CENTER DRIVE, THOUSAND OAKS, CA, 91320-1799, US

(73) Assignee: Amgen Inc.

(21) Appl. No.: 12/685,462

(22) Filed: Jan. 11, 2010

Related U.S. Application Data
(62) Division of application No. 11/780,942, filed on Jul. 20, 2007, now abandoned.
(60) Provisional application No. 60/832,541, filed on Jul. 21, 2006.

(51) Int. Cl. G01N 33/00

(52) U.S. Cl. 436/86

(57) ABSTRACT

The present invention relates in general to polypeptides having reduced susceptibility to oxidation, methods of selecting or making the polypeptides and methods of use thereof. The polypeptides having reduced susceptibility to oxidation are modified by amino acid substitution, deletion or insertion to confer reduced susceptibility to oxidation, thereby decreasing degradation of the polypeptide and extending the shelf-life and biological activity of the polypeptide under typical storage, handling and use conditions.

This application is a divisional of U.S. application Ser. No. 11/780,942 filed Jul. 20, 2007, which claims the priority benefit of U.S. Provisional Patent Application No. 60/832,541, filed Jul. 21, 2006, hereby incorporated by reference in their entirety.

### FIELD OF THE INVENTION

The present invention relates in general to polypeptides having reduced susceptibility to oxidation, methods of selecting or making the polypeptides and methods of use thereof. The polypeptides having reduced susceptibility to oxidation are modified by amino acid substitution, deletion or insertion to confer reduced susceptibility to oxidation, thereby decreasing degradation of the polypeptide and extending the shelf-life and biological activity of the polypeptide under typical storage, handling and use conditions.

### BACKGROUND OF THE INVENTION

Therapeutic proteins may at times during the course of manufacturing, handling, storage, and administration be exposed to visible light, fluorescent light, ultraviolet radiation, free radicals, and other factors which may cause oxidation. Several amino acids are susceptible to degradation, including cysteine, histidine, methionine, tyrosine and tryptophan. (Berlett et al., J. Biol. Chem. 272:20313-16, 1997). Oxidized protein may have altered structural properties and may lose biological activity. Oxidation of amino acids due to light is termed photooxidation. Photooxidation of proteins often leads to yellowing of the protein in solution.

Tryptophan is a highly photosensitive amino acid (Holt et al., Biochimica et Biophysica Acta 499:131-38, 1977) that is found within innumerable proteins. Photooxidation of tryptophan has been implicated in the development of yellow and brown cataracts in the eye (Holt et al., supra) and as a cause for the discoloration of bleached wool (Dyer et al., Photochemistry and Photobiology 82:551-57, 2006). Tryptophan residues are readily oxidized by hydrogen peroxide, atmospheric oxygen, photooxidation or by irradiation in the presence of oxygen (Kanner et al, J Agric Food Chem 35:71-76, 1987). Experiments in which tryptophan-containing peptides were irradiated (>295 nm) was reported to result in formation of kynurenine (Kyn), N′-formylkynurenine, glycine, serine, alanine, and aspartic acid (Holt et al., supra). Many tryptophan oxidation products are chromophore species with hues varying from pale yellow to brown.

Thus, there remains a need in the art to reduce the sensitivity of therapeutic proteins, including antibodies, to oxidation, thereby decreasing the rate of degradation of the pharmaceutical protein product, and improving the ability of the protein to maintain activity in environments of oxidative stress.

### SUMMARY OF THE INVENTION

The present invention relates in general to identification of oxidation-sensitive regions within polypeptides, including antibodies, methods of selecting or screening for such polypeptides with sensitivity to oxidation, methods of selecting polypeptides with reduced susceptibility to oxidation, methods of modifying the amino acid sequence of polypeptides to confer a reduced sensitivity to oxidation, methods of making and using the modified polypeptides and stable compositions, the selected/modified polypeptides and compositions containing them.

In one aspect, the invention contemplates a modified polypeptide, for example, an antibody, with reduced sensitivity to oxidation comprising one or more mutations in an oxidation-sensitive region of a parent polypeptide that confer reduced sensitivity to oxidation, particularly photooxidation, compared to the parent polypeptide.

In one embodiment, the “oxidation-sensitive region” comprises a surface-exposed region of a polypeptide of about 35, 30, 25, or 20, or less amino acids, that (a) contains at least one tryptophan and (b) lacks an oxidation-protective amino acid. In a related embodiment, the oxidation-sensitive region (a) contains two tryptophans separated by at least one, but less than about 15, or less than about 10, amino acids, and (b) lacks an oxidation-protective amino acid, preferably methionine, within about 10 amino acids of one of the tryptophans. Although the two tryptophans may be 15 amino acids apart in a linear amino acid sequence, folding (e.g. in a loop structure) may cause the tryptophans to be relatively close in the three-dimensional spatial structure.

Exemplary surface exposed regions include a surface-exposed loop, e.g. in a CDR-like geometry. Additional exemplary surface-exposed regions include protein-protein binding domains, catalytic domains, protein-nucleotide binding domains, extracellular domains of receptors, Ig-like domains, and Fc domains. In one embodiment, the oxidation-sensitive region consists essentially of the FR3, CDR3 and FR4 of the heavy chain of an antibody. In exemplary embodiments, the antibody comprises a framework derived from IgG1 or IgG2.

In another aspect, the invention provides methods of screening for polypeptides susceptible to oxidation by identifying oxidation-sensitive regions, and computer apparatus or programs that carry out such screening.

In a further aspect, the invention provides methods for modifying polypeptides susceptible to oxidation by making a mutation within the oxidation-sensitive region. The mutation may be an amino acid insertion, deletion or substitution. A mutation within the oxidation-sensitive region that confers reduced susceptibility to oxidation may be designed according to certain “oxidation reduction criteria,” including “photooxidation reduction criteria” which include: deletion of a tryptophan, substitution of a tryptophan with a different amino acid, insertion of an oxidation-protective amino acid, or substitution of an amino acid with an oxidation-protective amino acid. Exemplary oxidation-protective amino acids include amino acids that are easily oxidizable, including methionine, cysteine, histidine, phenyalanine, tyrosine, arginine, lysine and proline.

In exemplary embodiments where there are two tryptophans in the oxidation-sensitive region, oxidation reduction criteria may include any one of the following: (a) a trytophan is removed or substituted with a different amino acid; (b) a methionine is inserted or substituted between said two tryptophans; (c) a methionine is inserted or substituted up to 10 amino acids, or preferably within 6 or 4 amino acids, N-terminal of the N-terminal of said two tryptophan; or (d) a methionine is inserted or substituted up to 10 amino acids, or preferably within 6 or 4 amino acids, C-terminal of the C-terminal of said two tryptophan. In exemplary embodiments, the substitution is a conservative substitution, wherein the polypeptide retains the biological activity of the parent polypeptide. In a further exemplary embodiments, the substitution is a non-conservative substitution, wherein the polypeptide retains the biological activity of the parent polypeptide.

In another aspect, the invention provides a method of making the modified polypeptide or antibody comprising the steps of: (a) making a mutation in an oxidation-sensitive region of a parent polypeptide that confers reduced sensitivity to oxidation and (b) testing the mutated polypeptide from step (a) for sensitivity to oxidation. Testing for sensitivity to oxidation comprises exposing the modified polypeptide to oxidative stress, e.g. visible, fluorescent and/or UV light for photooxidation testing, and analyzing for oxidation of amino acids by techniques commonly used in the art, including, but not limited to, mass spectrometry, NMR, X-ray crystallography and visual inspection. Methods of producing the modified polypeptide are well known in the art and include construction of appropriate encoding nucleic acids and expressing such nucleic acids in suitable host cells, which may include steps of culturing the host cells in medium under suitable conditions, and purification of the desired modified polypeptide from the host cell or its culture medium.

Another aspect of the invention provides a method of selecting a polypeptide or antibody with reduced sensitivity to oxidation comprising the steps of: (a) analyzing the amino acid sequence of a surface-exposed region of a candidate polypeptide or antibody for the presence or absence of at least one, or at least two, surface-exposed tryptophans and the presence or absence of an oxidation-protective amino acid, wherein if there are two tryptophans they are in spatial proximity such that the residues are close enough to interact in an oxidative reaction; (b) selecting the candidate polypeptide or antibody as likely to have reduced sensitivity to oxidation if it meets the oxidation reduction criteria described herein, and, (c) optionally testing the candidate polypeptide or antibody for sensitivity to oxidation.

In one embodiment, oxidation reduction criteria, including photooxidation reduction criteria, further comprises selecting a polypeptide having a surface-exposed region of about 35, 30, 25, or 20 or less amino acids that (a) comprises only one tryptophan, or (b) comprises two tryptophans separated by more than 15 amino acids and spatially distant from each other in the three-dimensional polypeptide structure, or (c) if there are two tryptophans, having an oxidation protective amino acid, preferably within 10, 8, 6, or 4 amino acids of one of the tryptophans.

In a related aspect, the invention provides a computer program or apparatus programmed to carry out the steps of analyzing the amino acid sequence of a surface exposed region for the presence or absence of at least one, or at least two, surface-exposed tryptophans and the presence or absence of an oxidation-protective amino acid, and selecting the candidate polypeptide as likely to be susceptible to oxidation, or likely to have reduced sensitivity to oxidation.

In a further aspect, the invention provides a polypeptide or antibody produced or selected by any of the preceding methods of the invention.

The invention further contemplates a method of protecting a polypeptide or antibody from oxidation comprising the steps of: (a) analyzing the amino acid sequence of a surface-exposed region of a candidate polypeptide or antibody for the presence or absence of at least one, or at least two, tryptophans and the presence or absence of an oxidation-protective amino acid, and (b) storing in an oxidation-protective environment. A oxidation protective environment includes, but is not limited to, dark or opaque containers, or an oxygen free environment such as storage in the presence of noble gases. Exemplary noble gases include nitrogen, helium, argon and neon.

In yet another aspect of the invention, a pharmaceutical composition is provided comprising any one of the aforementioned polypeptides or antibodies and a pharmaceutically suitable carrier, excipient or diluent. Compositions containing the polypeptides of the invention exhibit reduced susceptibility to oxidation and preferably have prolonged shelf-life of at least 3, 6, 9, 12, 15, 18, 21 or 24 months when stored under typical conditions of exposure to ambient light. A stable aqueous solution should preferably retain its original clarity and color throughout its shelf life, and preferably over a relatively wide temperature range such as about 4° C. to about 37° C. and under exposure to ambient light.

A related aspect of the invention provides methods of using the pharmaceutical compositions comprising any of the preceding therapeutic polypeptides or antibodies by administering therapeutically effective amounts to a subject in need thereof.

Additional features and variations of the invention will be apparent to those skilled in the art from the entirety of this application, and all such features are intended as aspects of the invention.

### BRIEF DESCRIPTION OF THE DRAWINGS

FIG. 1 depicts the chemical pathway of tryptophan oxidation to isophenoxazine.

FIG. 2 is a mass spectrometry analysis showing the oxidation of tryptophan to alanine in a peptide derived from the CDR3-FR4 region of an antibody.

FIG. 3 is a comparison of the amino acid sequences of the CDR3-FR4 regions of antibodies that are considered susceptible to yellowing (i.e. in aqueous solution, the antibodies oxidize further and take on a darker hue upon exposure to light) (SEQ ID NOs:1-6) or “non-yellowing” (i.e. may have a pale yellow color, but do not oxidize or discolor further or are less susceptible to oxidation and discoloration) (SEQ ID NOs:7-11).

### DETAILED DESCRIPTION

An “immunoglobulin” or “native antibody” is a tetrameric glycoprotein composed of two identical pairs of polypeptide chains (two “light” and two “heavy” chains). The amino-terminal portion of each chain includes a “variable” (“V”) region of about 100 to 110 or more amino acids primarily responsible for antigen recognition. Within this variable region, the “hypervariable” region or “complementarity determining region” (CDR) has been determined by one method to consist of residues 24-34 (CDRL1), 50-56 (CDRL2) and 89-97 (CDRL3) in the light chain variable domain and 31-35 (CDRH1), 50-65 (CDRH2) and 95-102 (CDRH3) in the heavy chain variable domain, according to the amino acid numbering system as described by Kabat et al., Sequences of Proteins of Immunological Interest, 5th Ed. Public Health Service, National Institutes of Health, Bethesda, Md. (1991) and/or those residues from a hypervariable loop (i.e., residues 26-32 (CDRL1), 50-52 (CDRL2) and 91-96 (CDRL3) in the light chain variable domain and 26-32 (CDRH1), 53-55 (CDRH2) and 96-101 (CDRH3) in the heavy chain variable domain as described by Chothia et al., J. Mol. Biol. 196: 901-917 (1987). It is commonly understood in the art that the amino acid boundaries of the above regions may vary between antibodies. The carboxy-terminal portion of each antibody heavy chain defines a constant region primarily responsible for effector function.

Oxidation of amino acids within antibodies may lead to oxidation products in the solution, or present within the antibody sequence, that emit a pale yellow cast such that the antibody solution emits a slight yellow color. Further yellowing and darkening of antibody solutions that occurs after the initial purification of the antibodies, particularly after prolonged exposure to light, is indicative of additional oxidation. An antibody that after purification continues to oxidize and yellow is referred to as yellowing antibody or antibody susceptible to yellowing. An antibody that, either before or after purification, may have a slight yellow color, but does not continue to yellow after purification is referred to as a non-yellowing antibody or an antibody less susceptible to yellowing.

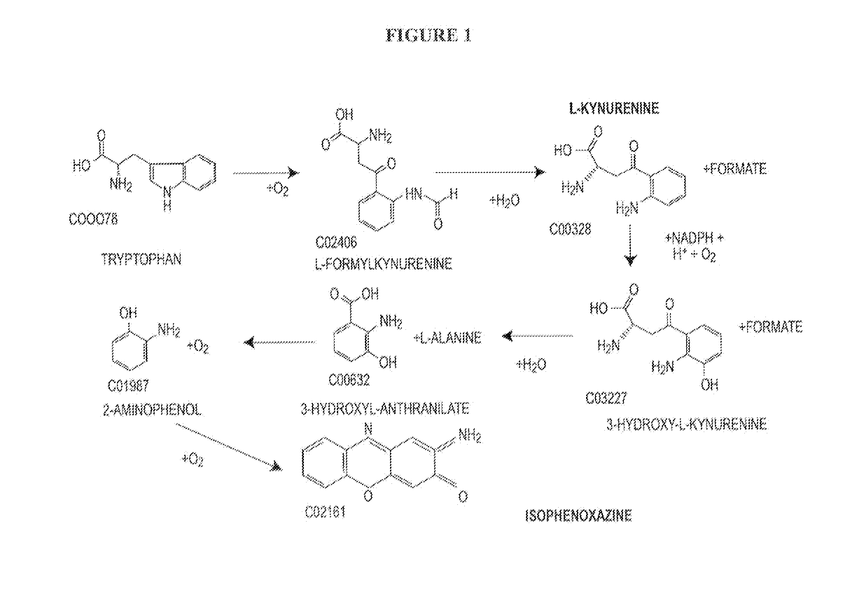

Not wishing to be bound by theory, the data herein indicate that such yellowing, in susceptible antibodies, is likely due to extensive oxidation of a tryptophan in the surface-exposed CDRH3 region of an immunoglobulin. See FIG. 1. The oxidation process causes further degradation of the tryptophan to an alanine, and the cleaved aromatic tryptophan side chain forms a 2-aminophenol (2-AP) product (Rogers et al., Proc Soc Exp Biol Med. 178:275-8, 1985) that further reacts to form a isophenoxazine (APX) (Simandi et al., Dalton Trans. 7:1056-60, 2004) which has a dark yellow hue.

In many IgG1 or IgG2 antibodies, the first amino acid in the FR4 region is also a tryptophan. Analysis of an immunoglobulin that lacks a tryptophan in CDR3 but has the tryptophan in FR4 indicates that this tryptophan in FR4 is oxidized to 3-hydroxy-L-kynurenine but that oxidation does not proceed further to alanine.

As described in further detail in the examples, comparison of the amino acid sequences in the FR3-CDR3-FR4 region of immunoglobulins revealed that the presence of a tryptophan within 10-15 amino acids of (but not adjacent to) the initial tryptophan in FR4 was correlated with susceptibility to photooxidation. The analysis also revealed that the presence of a methionine within 8 amino acids of one of these tryptophans was correlated with reduced susceptibility to photooxidation.

The term “modified polypeptide” as used herein refers to a polypeptide that has been artificially modified by mutation in an oxidation-sensitive region of a polypeptide, wherein the mutation is an insertion, deletion or substitution of an amino acid. Mutations that arise naturally without manipulation of the protein sequence or the encoding nucleic acid sequence are thus excluded from “modified polypeptide”. A “parent polypeptide” as used herein refers to the polypeptide sequence of the polypeptide prior to being modified by mutation.

The term “therapeutic polypeptide” refers to any polypeptide or fragment thereof administered to correct a physiological defect including inborn genetic errors, to replace a protein that is not expressed or expressed at low level in a subject or to alleviate, prevent or eliminate a disease state or condition in a subject. The term “therapeutic efficacy” refers to ability to of the therapeutic polypeptide to (a) prevent the development of a disease state or pathological condition, either by reducing the likelihood of or delaying onset of the disease state or pathological condition or (b) reduce or eliminate some or all of the clinical symptoms associated with the disease state or pathological condition.

The term “reduced sensitivity to photooxidation” as used herein refers to the reduced ability of the amino acids comprising the protein to be susceptible to oxidizing by light sources. Exemplary photooxidizing agents include UV light (e.g., far UV (200-10 nm) and near UV (380-200 nm), which may be divided into UVA (380-315 nm), UVB (315-280 nm) and UVC (<280 nm)) and visible light (400-800 nm) including fluorescent light. The term “reduced sensitivity to oxidation” as used herein refers to the reduced ability of the amino acids comprising the protein to be susceptible to oxidizing by light sources and any other source of oxidation, including but not limited to, ionizing radiation, oxygen radicals, metal ion and other oxidizing species known in the art. Reduced sensitivity to oxidation or photooxidation may be measured using a variety of readings, including mass spectrometry, NMR, and visual color measurement. Readouts taken for the modified polypeptide will show less oxidation as measured by procedures known to one of ordinary skill in the art when compared to the parent polypeptide.

The term “antibody” is used in the broadest sense and includes fully assembled antibodies, monoclonal antibodies, polyclonal antibodies, multispecific antibodies (e.g., bispecific antibodies), antibody fragments that can bind antigen (e.g., Fab′, F′(ab)₂, Fv, single chain antibodies, diabodies), and recombinant peptides comprising the forgoing as long as they exhibit the desired biological activity. Antigen-binding portions may be produced by recombinant DNA techniques or by enzymatic or chemical cleavage of intact antibodies. Antibody fragments or antigen-binding portions include, inter alia, Fab, Fab′, F(ab′)₂, Fv, domain antibody (dAb), complementarity determining region (CDR) fragments, single-chain antibodies (scFv), single chain antibody fragments, chimeric antibodies, diabodies, triabodies, tetrabodies, minibody, linear antibody; chelating recombinant antibody, a tribody or bibody, an intrabody, a transbody, a nanobody, a small modular immunopharmaceutical (SMIP), a antigen-binding-domain immunoglobulin fusion protein, a camelized antibody, a V_{HH} containing antibody, or a variant or a derivative thereof, and polypeptides that contain at least a portion of an immunoglobulin that is sufficient to confer specific antigen binding to the polypeptide, such as a CDR sequence, as long as the antibody retains the desired biological activity.

Amino Acid Oxidation

The amino acids that are particularly susceptible to oxidation include methionine, cysteine, histidine, and tyrosine; however, oxidation products have also been observed for proline, lysine, and arginine (Amici et al., J Biol. Chem. 264:3341-46. 1989; Stadtman, Free Radic Biol Med. 9:315-25, 1990) Amino acid oxidation is typically initiated by the presence of OH or O₂⁻reactive species, which may be generated by ionizing radiation (Berlett et al., supra). Oxygen reactive species target the protein backbone, stealing a hydrogen atom from an amino acid sidegroup to form a carbon radical. Formation of this carbon radical may ultimately lead to weakened peptide bonds subject to cleavage and protein fragmentation.

Cysteine and methionine residues are highly sensitive to oxidation and are rapidly converted to disulfides (Cys) and sulfoxide and sulfone residues (Met) in the presence of oxidizing species. These oxidation reactions are reversible and the oxidation of methionine is believed to not alter protein function. As such, methionine is hypothesized to be an internal protein anti-oxidant (Levine et al., Proc. Natl. Acad. Sci. USA 93:15036-40, 1996; Atmaca G., Yonsei Med J. 5:776-88, 2004).

Tryptophan (Trp) residues are oxidizable by peroxide and other oxygen species as well as by ionizing radiation. Tryptophan residues are typically not oxidized by metal-catalyzed oxidation because Trp is not likely a site for metal ion binding (Finley et al., Prot Sci 7:2391-97, 1998). Tryptophan oxidation products are themselves photosensitizers capable of generating reactive oxygen species (ROS) and can perpetuate the oxidation of other amino acids within a protein.

Several tryptophan oxidation products including hydroxytryptophans(HTRP), N-formyl-L-kynurenine (NFK), L-kynurenine (KYN), and 3-hydroxy-L-kynurenine (3-OH-KYN) have been identified by characteristic absorbance and fluorescence spectra (van Heyningen, Nature 230:393-94, 1971; Holt et al., supra; Maskos et al., Arch Biochem Biophys 296:514-20, 1992; Sen et al., Photochem Photobiol. 55:753-64, 1992). These products typically demonstrate a light pale yellow having an absorbance of approximately 360-380 nm. Additional downstream tryptophan oxidation products include glycine, serine, alanine and aspartic acid (Holt et al., supra). 2-aminophenol (2-AP), isophenoxazine (APX) are generated by further oxidation of 3-hydroxykynurenine 3-OH-KYN (FIG. 1). The oxidation products 2-AP and APX exhibit a darker yellow color having an absorbance of approximately 425-430 nm (Zhao et al., Appl Environ Microbiol, 66:2336-2342, 2000; Spiess et al., Appl Environ Microbiol, 64:446-452, 1998; Oancea et al., Central Eur J Chem, 1:233-241, 2003).

Amino acid oxidation may be measured using techniques standard in the art, including mass spectrometry, nuclear magnetic resonance (NMR), X-ray crystallography and visual inspection. Generally, readouts of the photooxidized polypeptide and modified polypeptide of the invention are compared to a standard having a known readout. Methods for measuring amino acid oxidation by mass spectrometry are described, for example, in Holt et al (Biochemica et Biophysica Acta 499:131-38, 1977), Finley et al. (Prot Sci 7:2391-97, 1998) and U.S. Pat. No. 6,096,556. Analysis of amino acid oxidation by NMR may be performed as described in Sala et al. (Eur. J. Biochem. 271:2841-2852, 2004) and Fu et al. (J Biol. Chem. 279:6209-12, 2004). X-ray crystallographic analysis of proteins mat be performed as described in Zhu et al. (Proc Natl Acad Sci USA. 101:2247-52, 2004), U.S. Pat. No. 6,860,940, and U.S. Pat. No. 6,069,235. The above references are herein incorporated by reference in their entirety.

Antibodies

The term “antibody” is used in the broadest sense and includes fully assembled antibodies, monoclonal antibodies, polyclonal antibodies, multispecific antibodies (e.g., bispecific antibodies), antibody fragments that can bind antigen (e.g., Fab′, F′(ab)₂, Fv, single chain antibodies, diabodies), and recombinant peptides comprising the forgoing as long as they exhibit the desired biological activity. Multimers or aggregates of intact molecules and/or fragments, including chemically derivatized antibodies, are contemplated. Antibodies of any isotype class or subclass, including IgG, IgM, IgD, IgA, and IgE, IgG1, IgG2, IgG3, IgG4, IgA1 and IgA2, are contemplated. Different isotypes have different effector functions; for example, IgG1 and IgG3 isotypes have antibody-dependent cellular cytotoxicity (ADCC) activity.

The term “monoclonal antibody” as used herein refers to an antibody obtained from a population of substantially homogeneous antibodies, i.e., the individual antibodies comprising the population are identical except for possible naturally occurring mutations or alternative post-translational modifications that may be present in minor amounts, whether produced from hybridomas or recombinant DNA techniques. Nonlimiting examples of monoclonal antibodies include murine, chimeric, humanized, or human antibodies, or variants or derivatives thereof. Humanizing or modifying antibody sequence to be more human-like is described in, e.g., Jones et al., Nature 321:522 525 (1986); Morrison et al., Proc. Natl. Acad. Sci., U.S.A., 81:6851 6855 (1984); Morrison and Oi, Adv. Immunol., 44:65 92 (1988); Verhoeyer et al., Science 239:1534 1536 (1988); Padlan, Molec. Immun. 28:489 498 (1991); Padlan, Molec. Immunol. 31(3):169 217 (1994); and Kettleborough, C. A. et al., Protein Eng. 4(7):773 83 (1991); Co, M. S., et al. (1994), J. Immunol. 152, 2968-2976); Studnicka et al. Protein Engineering 7: 805-814 (1994); each of which is incorporated herein by reference. One method for isolating human monoclonal antibodies is the use of phage display technology. Phage display is described in e.g., Dower et al., WO 91/17271, McCafferty et al., WO 92/01047, and Caton and Koprowski, Proc. Natl. Acad. Sci. USA, 87:6450-6454 (1990), each of which is incorporated herein by reference. Another method for isolating human monoclonal antibodies uses transgenic animals that have no endogenous immunoglobulin production and are engineered to contain human immunoglobulin loci. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); WO 91/10741, WO 96/34096, WO 98/24893, or U.S. patent application publication nos. 20030194404, 20030031667 or 20020199213; each incorporated herein by reference.

Antibody fragments may be produced by recombinant DNA techniques or by enzymatic or chemical cleavage of intact antibodies. “Antibody fragments” comprise a portion of an intact full length antibody, preferably the antigen binding or variable region of the intact antibody, and include multispecific (bispecific, trispecific, etc.) antibodies formed from antibody fragments. Nonlimiting examples of antibody fragments include Fab, Fab′, F(ab′)₂, Fv [variable region], domain antibodies (dAb, containing a V_{H} domain) [Ward et al., Nature 341:544-546, 1989], complementarity determining region (CDR) fragments, single-chain antibodies (scFv, containing V_{H} and V_{L} domains on a single polypeptide chain) [Bird et al., Science 242:423-426, 1988, and Huston et al., Proc. Natl. Acad. Sci. USA 85:5879-5883, 1988, optionally including a polypeptide linker; and optionally multispecific, Gruber et al., J. Immunol. 152: 5368 (1994)], single chain antibody fragments, diabodies (V_{H} and V_{L} domains on a single polypeptide chain that pair with complementary V_{L} and V_{H} domains of another chain) [EP 404,097; WO 93/11161; and Hollinger et al., Proc. Natl. Acad. Sci. USA, 90:6444-6448 (1993)], triabodies, tetrabodies, minibodies (scFv fused to CH₃ via a peptide linker (hingeless) or via an IgG hinge) [Olafsen, et al., Protein Eng Des Sel. 2004 April; 17(4):315-23], linear antibodies (tandem Fd segments (V_{H}—C_{H}1-V_{H}—C_{H}1) [Zapata et al., Protein Eng., 8(10):1057-1062 (1995)]; chelating recombinant antibodies (crAb, which can bind to two adjacent epitopes on the sane antigen) [Neri et al., J Mol. Biol. 246:367-73, 1995], bibodies (bispecific Fab-scFv) or tribodies (trispecific Fab-(scFv)(2)) [Schoonjans et al., J. Immunol. 165:7050-57, 2000; Willems et al., J Chromatogr B Analyt Technol Biomed Life Sci. 786:161-76, 2003], intrabodies [Biocca, et al., EMBO J. 9:101-108, 1990; Colby et al., Proc Natl Acad Sci USA. 101:17616-21, 2004] which may also comprise cell signal sequences which retain the antibody intracellularly [Mhashilkar et al, EMBO J. 14:1542-51, 1995; Wheeler et al., FASEB J. 17:1733-5, 2003], transbodies (cell-permeable antibodies containing a protein transduction domain (PTD) fused to scFv [Heng et al., Med. Hypotheses. 64:1105-8, 2005], nanobodies (approximately 15 kDa variable domain of the heavy chain) [Cortez-Retamozo et al., Cancer Research 64:2853-57, 2004], small modular immunopharmaceuticals (SMIPs) [WO03/041600, U.S. Patent publication 20030133939 and US Patent Publication 20030118592], an antigen-binding-domain immunoglobulin fusion protein, a camelized antibody (in which V_{H} recombines with a constant region that contains hinge, CH1, CH2 and CH3 domains) [Desmyter et al., J. Biol. Chem. 276:26285-90, 2001; Ewert et al., Biochemistry 41:3628-36, 2002; U.S. Patent Publication Nos. 20050136049 and 20050037421], a VHH containing antibody, heavy chain antibodies (HCAbs, homodimers of two heavy chains having the structure H₂L₂), or variants or derivatives thereof, and polypeptides that contain at least a portion of an immunoglobulin that is sufficient to confer specific antigen binding to the polypeptide, such as a CDR sequence, as long as the antibody retains the desired biological activity.

The term “variant” when used in connection with antibodies refers to polypeptide sequence of an antibody that contains at least one amino acid substitution, deletion, or insertion in the variable region or the portion equivalent to the variable region, provided that the variant retains the desired binding affinity or biological activity. In addition, the antibodies of the invention may have amino acid modifications in the constant region to modify effector function of the antibody, including half-life or clearance, ADCC and/or CDC activity. Such modifications can enhance pharmacokinetics or enhance the effectiveness of the antibody in treating cancer, for example. See Shields et al., J. Biol. Chem., 276(9):6591-6604 (2001), incorporated by reference herein in its entirety. In the case of IgG1, modifications to the constant region, particularly the hinge or CH2 region, may increase or decrease effector function, including ADCC and/or CDC activity. In other embodiments, an IgG2 constant region is modified to decrease antibody-antigen aggregate formation. In the case of IgG4, modifications to the constant region, particularly the hinge region, may reduce the formation of half-antibodies.

The term “derivative” when used in connection with antibodies refers to antibodies covalently modified by conjugation to therapeutic or diagnostic agents, labeling (e.g., with radionuclides or various enzymes), covalent polymer attachment such as pegylation (derivatization with polyethylene glycol) and insertion or substitution by chemical synthesis of non-natural amino acids. Derivatives of the invention will retain the binding properties of underivatized molecules of the invention. Conjugation of cancer-targeting antibodies to cytotoxic agent, for example, radioactive isotopes (e.g., I131, I125, Y90 and Re186), chemotherapeutic agents, or toxins, may enhance destruction of cancerous cells.

Methods for making bispecific or other multispecific antibodies are known in the art and include chemical cross-linking, use of leucine zippers [Kostelny et al., J. Immunol. 148:1547-1553, 1992]; diabody technology [Hollinger et al., Proc. Natl. Acad. Sci. USA 90:6444-48, 1993]; scFv dimers [Gruber et al., J. Immunol. 152: 5368, 1994], linear antibodies [Zapata et al., Protein Eng. 8:1057-62, 1995]; and chelating recombinant antibodies [Neri et al., J Mol. Biol. 246:367-73, 1995].

Thus, a variety of compositions comprising one, two, and/or three CDRs of a heavy chain variable region or a light chain variable region of an antibody may be generated by techniques known in the art.

Modification of Polypeptides

The polypeptides or antibodies of the invention are modified by techniques well-known to one of ordinary skill in the art. Potential mutations include insertion, deletion or substitution of one or more residues. Insertions or deletions are preferably in the range of about 1 to 5 amino acids, more preferably 1 to 3, and most preferably 1 or 2 amino acids. The variation may be introduced by systematically making substitutions of amino acids in an antibody polypeptide molecule using recombinant DNA techniques well known in the art and assaying the resulting recombinant variants for activity. Nucleic acid alterations can be made at sites that differ in the nucleic acids from different species (variable positions) or in highly conserved regions (constant regions). Methods for altering antibody sequences and expressing antibody polypeptide compositions useful in the invention are described in greater detail below.

Substitution refers to a modified polypeptide with at least one amino acid residue in the polypeptide molecule removed and a different residue inserted in its place. Substitution includes substitution with alanine, a conservative substitution, or a non-conservative substitution. Conservative substitutions involve replacing an amino acid with another member of its class. Non-conservative substitutions involve replacing a member of one of these classes with a member of another class. Substitutional mutagenesis within any of the surface exposed regions of a polypeptide, such as the hypervariable or CDR regions or framework regions of an antibody, is contemplated. Further substitutions include, in the case of an antibody, replacement with a corresponding amino acid residue at the same position from a different IgG subclass (e.g. replacing an IgG1 residue with a corresponding IgG2 residue at that position).

Conservative amino acid substitutions are made on the basis of similarity in polarity, charge, solubility, hydrophobicity, hydrophilicity, and/or the amphipathic nature of the residues involved. For example, nonpolar (hydrophobic) amino acids include alanine (Ala, A), leucine (Leu, L), isoleucine (Ile, I), valine (Val, V), proline (Pro, P), phenylalanine (Phe, F), tryptophan (Trp, W), and methionine (Met, M); polar neutral amino acids include glycine (Gly, G), serine (Ser, S), threonine (Thr, T), cysteine (Cys, C), tyrosine (Tyr, Y), asparagine (Asn, N), and glutamine (Gln, Q); positively charged (basic) amino acids include arginine (Arg, R), lysine (Lys, K), and histidine (H is, H); and negatively charged (acidic) amino acids include aspartic acid (Asp, D) and glutamic acid (Glu, E).

Techniques for cloning and expressing nucleotide and polypeptide sequences are well-established in the art [see e.g. Sambrook et al., Molecular Cloning: A Laboratory Manual, 2nd Edition, Cold Spring Harbor, N.Y. (1989)]. For example, the nucleic acid encoding a polypeptide or a modified polypeptide is isolated and inserted into a replicable vector for further cloning (amplification of the DNA) or for expression. DNA encoding the monoclonal antibody is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more selective marker genes, an enhancer element, a promoter, and a transcription termination sequence.

Alternatively, or in addition, it may be beneficial to analyze a crystal structure of the polypeptide or antibody to identify amino acids in surface exposed regions, or to use computer software to model such surface exposed regions, to determine amino acid oxidation. Such exposed residues and neighboring residues are candidates for substitution according to the techniques elaborated herein. Once such modified polypeptides are generated, the panel of modified polypeptides is subjected to screening as described herein.

Any combination of deletion, insertion, and substitution is made to arrive at the final construct, provided that the final construct possesses the desired characteristics.

Formulation of Pharmaceutical Compositions

To administer modified or selected polypeptides, including antibodies, of the invention to human or test animals, it is preferable to formulate the modified polypeptides in a composition comprising one or more pharmaceutically acceptable carriers. The phrase “pharmaceutically or pharmacologically acceptable” refer to molecular entities and compositions that do not produce allergic, or other adverse reactions when administered using routes well-known in the art, as described below. “Pharmaceutically acceptable carriers” include any and all clinically useful solvents, dispersion media, coatings, antibacterial and antifungal agents, isotonic and absorption delaying agents and the like.

Examples include, but are not limited to, any of a number of standard pharmaceutical carriers such as sterile phosphate buffered saline solutions, bacteriostatic water, and the like. A variety of aqueous carriers may be used, e.g., water, buffered water, 0.4% saline, 0.3% glycine and the like, and may include other proteins for enhanced stability, such as albumin, lipoprotein, globulin, etc., subjected to mild chemical modifications or the like.

Exemplary polypeptide concentrations in the formulation may range from about 0.1 mg/ml to about 180 mg/ml or from about 0.1 mg/mL to about 50 mg/mL, or from about 0.5 mg/mL to about 25 mg/mL, or alternatively from about 2 mg/mL to about 10 mg/mL. An aqueous formulation of the polypeptide may be prepared in a pH-buffered solution, for example, at pH ranging from about 4.5 to about 6.5, or from about 4.8 to about 5.5, or alternatively about 5.0. Examples of buffers that are suitable for a pH within this range include acetate (e.g. sodium acetate), succinate (such as sodium succinate), gluconate, histidine, citrate and other organic acid buffers. The buffer concentration can be from about 1 mM to about 200 mM, or from about 10 mM to about 60 mM, depending, for example, on the buffer and the desired isotonicity of the formulation.

A tonicity agent, which may also stabilize the polypeptide, may be included in the formulation. Exemplary tonicity agents include polyols, such as mannitol, sucrose or trehalose. Preferably the aqueous formulation is isotonic, although hypertonic or hypotonic solutions may be suitable. Exemplary concentrations of the polyol in the formulation may range from about 1% to about 15% w/v.

A surfactant may also be added to the polypeptide formulation to reduce aggregation of the formulated polypeptide and/or minimize the formation of particulates in the formulation and/or reduce adsorption. Exemplary surfactants include nonionic surfactants such as polysorbates (e.g. polysorbate 20, or polysorbate 80) or poloxamers (e.g. poloxamer 188). Exemplary concentrations of surfactant may range from about 0.001% to about 0.5%, or from about 0.005% to about 0.2%, or alternatively from about 0.004% to about 0.01% w/v.

In one embodiment, the formulation contains the above-identified agents (i.e. polypeptide, buffer, polyol and surfactant) and is essentially free of one or more preservatives, such as benzyl alcohol, phenol, m-cresol, chlorobutanol and benzethonium Cl. In another embodiment, a preservative may be included in the formulation, e.g., at concentrations ranging from about 0.1% to about 2%, or alternatively from about 0.5% to about 1%. One or more other pharmaceutically acceptable carriers, excipients or stabilizers such as those described in Remington's Pharmaceutical Sciences 16th edition, Osol, A. Ed. (1980) may be included in the formulation provided that they do not adversely affect the desired characteristics of the formulation. Acceptable carriers, excipients or stabilizers are nontoxic to recipients at the dosages and concentrations employed and include; additional buffering agents; co-solvents; antoxidants including ascorbic acid and methionine; chelating agents such as EDTA; metal complexes (e.g. Zn-protein complexes); biodegradable polymers such as polyesters; and/or salt-forming counterions such as sodium.

Therapeutic formulations of the polypeptide are prepared for storage by mixing the polypeptide having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, maltose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In one embodiment, a suitable formulation of the claimed invention contains an isotonic buffer such as a phosphate, acetate, or TRIS buffer in combination with a tonicity agent such as a polyol, Sorbitol, sucrose or sodium chloride which tonicifies and stabilizes. One example of such a tonicity agent is 5% Sorbitol or sucrose. In addition, the formulation could optionally include a surfactant such as to prevent aggregation and for stabilization at 0.01 to 0.02% wt/vol. The pH of the formulation may range from 4.5-6.5 or 4.5 to 5.5. Other exemplary descriptions of pharmaceutical formulations for antibodies may be found in US 2003/0113316 and U.S. Pat. No. 6,171,586, each incorporated herein by reference in its entirety.

The formulation herein may also contain more than one active compound as necessary for the particular indication being treated, preferably those with complementary activities that do not adversely affect each other. For example, it may be desirable to further provide an immunosuppressive agent. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Suspensions and crystal forms of polypeptides are also contemplated. Methods to make suspensions and crystal forms are known to one of skill in the art.

The formulations to be used for in vivo administration must be sterile. The compositions of the invention may be sterilized by conventional, well known sterilization techniques. For example, sterilization is readily accomplished by filtration through sterile filtration membranes. The resulting solutions may be

The process of freeze-drying is often employed to stabilize polypeptides for long-term storage, particularly when the polypeptide is relatively unstable in liquid compositions. A lyophilization cycle is usually composed of three steps: freezing, primary drying, and secondary drying; Williams and Polli, Journal of Parenteral Science and Technology, Volume 38, Number 2, pages 48-59 (1984). In the freezing step, the solution is cooled until it is adequately frozen. Bulk water in the solution forms ice at this stage. The ice sublimes in the primary drying stage, which is conducted by reducing chamber pressure below the vapor pressure of the ice, using a vacuum. Finally, sorbed or bound water is removed at the secondary drying stage under reduced chamber pressure and an elevated shelf temperature. The process produces a material known as a lyophilized cake. Thereafter the cake can be reconstituted prior to use. In one embodiment the lyophilization is performed in dark conditions.

The standard reconstitution practice for lyophilized material is to add back a volume of pure water (typically equivalent to the volume removed during lyophilization), although dilute solutions of antibacterial agents are sometimes used in the production of pharmaceuticals for parenteral administration; Chen, Drug Development and Industrial Pharmacy, Volume 18, Numbers 11 and 12, pages 1311-1354 (1992).

Excipients have been noted in some cases to act as stabilizers for freeze-dried products; Carpenter et al., Developments in Biological Standardization, Volume 74, pages 225-239 (1991). For example, known excipients include polyols (including mannitol, sorbitol and glycerol); sugars (including glucose and sucrose); and amino acids (including alanine, glycine and glutamic acid).

In addition, polyols and sugars are also often used to protect polypeptides from freezing and drying-induced damage and to enhance the stability during storage in the dried state. In general, sugars, in particular disaccharides, are effective in both the freeze-drying process and during storage. Other classes of molecules, including mono- and di-saccharides and polymers such as PVP, have also been reported as stabilizers of lyophilized products.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Sustained-release preparations may be prepared. Suitable examples of sustained-release preparations include semipermeable matrices of solid hydrophobic polymers containing the polypeptide, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release polypeptides for shorter time periods. When encapsulated polypeptides remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

The formulations of the invention may be designed to be short-acting, fast-releasing, long-acting, or sustained-releasing as described herein. Thus, the pharmaceutical formulations may also be formulated for controlled release or for slow release.

Specific dosages may be adjusted depending on conditions of disease, the age, body weight, general health conditions, sex, and diet of the subject, dose intervals, administration routes, excretion rate, and combinations of drugs. Any of the

The polypeptide may be administered by any suitable means, including parenteral, subcutaneous, intraperitoneal, intrapulmonary, and intranasal, and, if desired for local treatment, intralesional administration. Parenteral infusions include intravenous, intraarterial, intraperitoneal, intramuscular, intradermal or subcutaneous administration. In addition, the polypeptide is suitably administered by pulse infusion, particularly with declining doses of the polypeptide. Preferably the dosing is given by injections, most preferably intravenous or subcutaneous injections, depending in part on whether the administration is brief or chronic. Other administration methods are contemplated, including topical, particularly transdermal, transmucosal, rectal, oral or local administration e.g. through a catheter placed close to the desired site. Most preferably, the polypeptides of the invention are administered intravenously in a physiological solution at a dose ranging between 0.01 mg/kg to 100 mg/kg at a frequency ranging from daily to weekly to monthly (e.g. every day, every other day, every third day, or 2, 3, 4, 5, or 6 times per week), preferably a dose ranging from 0.1 to 45 mg/kg, 0.1 to 15 mg/kg or 0.1 to 10 mg/kg at a frequency of 2 or 3 times per week, or up to 45 mg/kg once a month.

The formulations may be stored in a prefilled syringe or vial and may be part of a kit.

In addition to formulation for pharmaceutical compounds, the polypeptides of the invention may be formulated in a photooxidation-protective environment. For example, the polypeptide may be formulated and stored, or in a dark or opaque container that is impervious to oxidative light. Further, the polypeptide may be stored in an oxygen free environment comprising stable, non-oxidizing gases, such as nitrogen, helium, argon and neon.

Also contemplated is formulation of the polypeptide of the invention in solution with oxidation-protective agents, including but not limited to free radical scavengers, such as mannitol, methionine, histidine, casein, ascorbic acid, and N-acetylcysteine.

Photostability Testing

Photostability of a formulation can be assessed by exposing the formulation to extreme conditions including lengthy exposure to UV-B radiation. While direct exposure to UV-B radiation is unlikely to be encountered during storage, handling, and administration of a polypeptide therapeutic, some UV-B radiation is often present and would lead to undesirable degradation of the polypeptide. The effectiveness of antioxidants in an aqueous formulation is assessed by exposing the formulations to UV-B and/or other sources of light including fluorescent room lighting, artificial daylight, UV-A light, UV-C light and near UV light.

The ICH guideline for photostability testing of a new drug substance gives recommendations for the light sources and exposure times that should be tested to ensure that such an exposure does not result in undesirable change. Two options for light sources are given by this guideline. The include (1) any light source that is designed to produce an output similar to the D65/ID65 emission standard such as an artificial daylight fluorescent lamp combining visible and ultraviolet outputs, xenon, or metal halide lamp; or (2) the sample is exposed to both a cool white fluorescent and near ultraviolet lamp. The cool white fluorescent lamp is set to produce an output similar to that specified in ISO 10977. The near UV fluorescent lamp has a spectral distribution from 320 nm to 400 nm with a maximum energy emission between 350 nm and 370 nm. A significant proportion of UV is in both bands of 320 nm to 360 nm and 360 nm to 400 nm.

Additional aspects and details of the invention will be apparent from the following examples, which are intended to be illustrative rather than limiting.

### Example 1

Tryptophan is known to be rapidly oxidized in the presence of reactive oxygen species. Studies have demonstrated that proteins lacking tryptophan or those having reduced numbers of tryptophan undergo decreased photooxidation (Dilley, K., Biochem J 133:821-26, 1973) Immunoglobulin proteins contain a number of tryptophan residues in both the constant regions and in the variable framework regions. Not all of these tryptophans are oxidized, and among those that are oxidized, they are degraded to different extents. To determine the degree of susceptibility of these tryptophans to photooxidation when the Ig molecules are kept in a solution in

Several differences in mass spectrometry between photooxidized Ig molecules and control molecules were discovered, leading to further investigation of which residues in the Ig molecule were degraded by the oxidation reaction. Mass spec of the Ig region demonstrated degradation of methionine 253 from the Ig heavy chain region and also revealed Trp degradation in CDR3 of the heavy chain variable region. Additional Trp degradation was detected in the CH3 Region of the Fc domain. Breakdown products detected include Kynurenine, N-formyl-Kynurenine, and 3 hydroxy-N-formyl Kynurenine which caused yellowing of the Ig solution.

These results indicate that the surface exposed Trp residues in IgG molecules can be susceptible to degradation upon exposure to intense light. Light exposure also induced oxidation of methionine residues to sulfoxide and sulfone forms. The generation of methionine sulfone could be indicative of the generation of free radicals after exposure to intense light.

### Example 2

The CDR3 loop comprises a flexible structure within the Ig molecule which allows the molecule to move in the biological milieu and bind to its cognate antigen. Crytallographic analysis demonstrates that residue 417 in the CDR3 loop is partially solvent exposed and potentially susceptible to photooxidation in the presence of light. Experiments were performed to assess the sensitivity of the CDR3 loop to photooxidation.

Evidence generated from a light stressed antibody sample (exposure to light for 21 months) indicated that APX, a yellow chromophore, is generated from the conversion of Trp to alanine (Ala). Further experiments demonstrated that chromatography by Protein A purification (MABSELECTT™, GE Healthcare, Piscataway, N.J.) and cation exchange chromatography (FRACTOGEL®, EMD Biosciences, Inc., San Diego, Calif.) removed either 2-AP, a colorless precursor of APX, or APX. After light exposure for 3-6 months, no color change or yellowing was observed in purified antibody samples at a concentration at 150 g/L in pre-formulation buffer. In addition, freshly purified antibody-containing solutions were colorless with no detectable APX and 2-AP. However, after a 2-week exposure to light, the solution turned yellow and 2-AP and APX were extracted by chromatography.

These results show that monoclonal antibodies containing tryptophan (Trp) residues in the CDR3 region are light sensitive. These results also demonstrate that although amino acid oxidation may take place during the purification methods and these byproducts are readily extracted, further amino acid photooxidation takes place to fully oxidize the susceptible amino acid residues, thereby causing yellowing of the antibody solutions.

A further experiment was carried out to compare the photooxidation of the antibody product HERCEPTIN® to other purified monoclonal antibody compositions. HERCEPTIN® and sample antibodies all at 21 mg/ml were dialyzed into buffer with three buffer changes and then exposed to deli case fluorescent light at 4° C. for 6 weeks. For mass spec analysis, 200 μg protein (1.9 μL) was mixed in denaturation buffer (478.1 μL, 6 M Gd—HCl, 0.1 M Tris base, 2 mM EDTA, pH 7.5) and reducing buffer (20 μL, 500 mM Dithiothreitol, DTT) vortexed briefly and cultured at 37° C. for 30 minutes. The samples were cooled to room temperature, spun down and mixed with alkylating reagent (50 μL, 500 mM Iodoactamide, IAM) and incubated at room temperature at dark for 30 minutes. Samples were buffer exchanged into 2× Digestion Buffer (100 mM Tris Base, 2 mM CaCl₂, 500 mM Gd—HCl, pH 7.7) using NAP-10 SPE desalting column and samples concentrated by collected fractionation (Amicon Ultra-4 (10 k MWCO)) tube, and spun at 3000 rpm to an average RCF of 1855 g for 20 minutes at 4° C. All retentate was recovered, and water added to bring 2× digestion buffer to lx, and trypsin added to make enzyme-to-substrate (E:S) ratio of 1:60. Samples were incubated at 37° C. in the dark for 16 hours. The digestion was terminated by adding 20 μL 5% (v/v) TFA to bring down solution pH below 2.

For mass spec analysis LC/MS, a reverse phase column was used (Polaris C18-A, 5 u, 250×2.0 mm, 55C) having a mobile phases of: (A) 0.1% (v/v) TFA in water; and, (B) 0.085% (v/v) TFA in 90% (v/v) in water. The gradient used was: 0% B (e.g., 100% A solution and 0% B) hold for 15 minutes, 0% B to 45% B solution over the course of 210 minutes, followed by column cleaning using 95% B solution, flush for 5 minutes and finally 0% B column equilibrium for 30 minutes. LC detection was at 214 nm and the MS mass scanning range was set at m/z 300 to m/z 2000 on Thermo Finnigan LCQ-Deca.

During dialysis experiments, no yellowing was found for HERCEPTIN® while sample antibodies became yellow after light exposure. Several other antibody products showed no yellowing changes during purification and production.

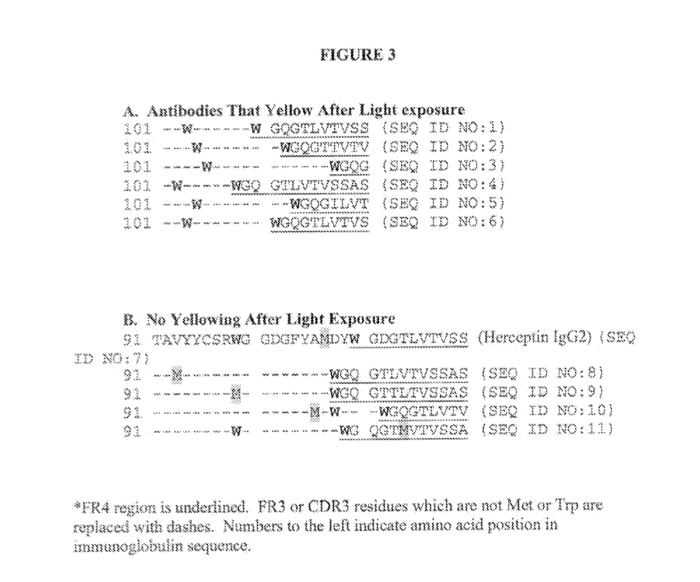

Comparison of the amino acid sequences in the CDR3 region of these monoclonal antibodies (Mab) was performed and yellowing Mab molecules compared to non-yellowing Mab in their amino acid sequences. It was found that yellowing antibodies typically demonstrated two Trp residues separated by 10-15 amino acids in the CDR3 region (amino acids 91 to 120) and there was no Met residue nearby to either Trp in primary sequence. For example, a Met greater than 8 amino acid residues in the C-terminal or N-terminal direction from the Trp in the CDR3 did not provide protection from photooxidation. Representative amino acid sequences and the relative positions of the Met and Trp are displayed in FIG. 3.

Analysis of non-yellowing monoclonal antibodies shows that non-yellowing antibodies typically demonstrated one Trp and one Met in the CDR3 region; but if there were two Trp in this region, the Met was positioned between the two Trp residues as in HERCEPTIN®. Additionally, if there were two Trp residues in this region, a Met could be placed either N-terminal or C-terminal to the two Trp residues. In non-yellowing antibodies, the distance between the Trp and Met residues in antibodies containing two Trp residues in the CDR3 region was approximately 6 amino acid residues.

Methionine has considerable potential to be oxidized compared to Trp oxidation. Trp residues in other regions, e.g., the heavy chain constant region, can be oxidized; however, these Trp residues have less surface exposure opportunities than CDR3 regions. Thus, the major reason for the non-yellowing Mab is that the Met in proximity to the Trp protects the oxidation of tryptophan in CDR3 region of Mab, thereby reducing the possibility of yellowing Mab production.

### Example 3

Most proteins undergo some degree of photooxidation. Many proteins having tryptophans that have undergone photooxidation will exhibit a pale yellow coloring due to the presence of kynurenine compounds or other oxidation products. This color is usually removed using standard protein purification methods such as cation or anion exchange columns. However, further oxidation of these proteins may take place during filling, storage, handling or therapeutic administration. The secondary oxidation of the tryptophan or kynurenine residues leads to a further yellowing of the protein solution. To determine if a secondary photooxidation reaction takes place in antibodies comprising tyrptophan residues in a surface exposed region of the polypeptide, the yellowing species were first removed and the protein sample subject to further light stress.

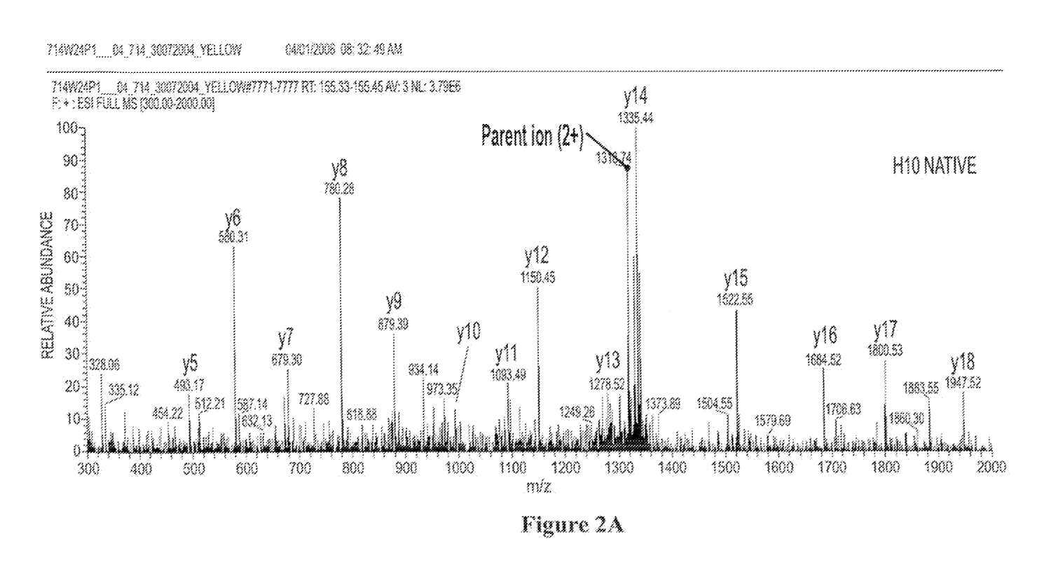

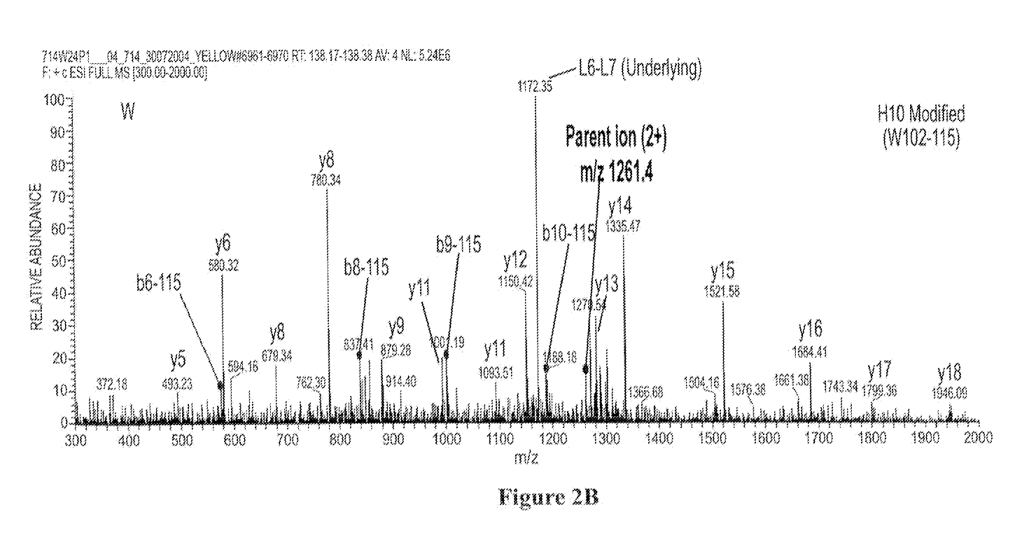

A peptide comprising a tryptophan in the CDR3 region and a tryptophan in the N-terminal end of the framework 4 (FR4) region of the IgG1 heavy chain variable region was modified by exposure to light at room temperature for one month, or kept in an environment protected from light oxidation. The samples were then compared by mass spectrometry as above to determine the end product of tryptophan oxidation. Comparison of the mass spectrometry data shows that the peptide modified by exposure to light (H10 Modified) is lighter in molecular weight by 115 (FIG. 2) compared to the native sequence (H10Native). This decrease in molecular weight is indicative of oxidation of the tryptophan in CDR3 to alanine, which results in the release of breakdown products 2-AP and APX. The tryptophan in the FR4 is maintained as a tryptophan.

These results demonstrate that additional photooxidation of surface-exposed tryptophan in the antibody CDR3 domain takes place upon continued exposure of the protein to light.

### Example 4

To further confirm that the tryptophan in CDR3 is highly susceptible to photooxidation and leads to the yellowing of antibodies in solution, an antibody product having a tryptophan in CDR3 and FR4 was mutated to have an alanine in place of the Trp in CDR3 (Trp→Ala). The wild type and mutant peptides were then exposed to light as described above. Visual observation of the solution color showed that the peptide sample having two tryptophans yellowed over time. The peptide having no Trp in CDR3 (Trp→Ala) and one Trp in FR4 did not yellow upon exposure to light.

These results confirm that two trytophans in close proximity, wherein one of the Trp is readily exposed on the surface of the protein in a surface-exposed region, are highly susceptible to photooxidation. Further, this susceptibility is reduced by modifying the peptide sequence to remove the surface-exposed tryptophan residue.

### Example 5

To confirm that the presence of a methionine within 10 amino acids of the trytophan in CDR3 or FR4 protects the tryptophan in CDR3 from photooxidation, an antibody product having two tryptophans in the CDR3-FR4 region is mutated to substitute a nonpolar amino acid with a Met. The wild type and mutant peptides are then exposed to light as described above. It is expected that visual observation of the solution color shows that the peptide sample having two tryptophans, without the methionine, yellows over time, and that the peptide that includes the methionine does not yellow upon exposure to light.

Thus, in a polypeptide having a surface-exposed region, such as the CDR3 region in an antibody, close proximity of a surface exposed Trp and non-exposed Trp sensitizes the polypeptide to photooxidation and degradation of the Trp to Ala. However, the presence of a methionine nearby reduces the susceptibility of the exposed Trp to photooxidation.

Thus, modification of surface-exposed regions to reduce susceptibility of the Trp to photooxidation, e.g. by addition, such as substitution or insertion, of methionine or modification of the Trp residue, provides stability to the protein composition during storage and handling of the composition.

Numerous modifications and variations in the invention as set forth in the above illustrative examples are expected to occur to those skilled in the art. Consequently only such limitations as appear in the appended claims should be placed on the invention

1-14. (canceled)

15. A method of making the modified polypeptide or antibody comprising the steps of:
(a) making a mutation in an oxidation-sensitive region of a parent polypeptide that confers reduced sensitivity to oxidation and
(b) testing the mutated polypeptide from step (a) for sensitivity to oxidation.

16. A method of selecting a polypeptide or antibody with reduced sensitivity to oxidation comprising the steps of:
(a) analyzing the amino acid sequence of a surface-exposed region of a candidate polypeptide/antibody for the presence or absence of at least two tryptophans and the presence or absence of an oxidation-protective amino acid,
(b) selecting the candidate polypeptide/antibody as likely to have reduced sensitivity to oxidation if it meets the oxidation reduction criteria, and
(c) optionally testing the candidate polypeptide/antibody for sensitivity to oxidation.

17. (canceled)

18. A method of protecting a polypeptide/antibody from oxidation comprising the steps of:
(a) analyzing the amino acid sequence of a surface-exposed region of a candidate polypeptide/antibody for the presence or absence of at least two tryptophans and the presence or absence of an oxidation-protective amino acid, and
(b) storing in an oxidation-protective environment.

19. The method of any one of claims 15, 16 or 18 wherein the oxidation is photooxidation.

20. (canceled)
